(19) United States
(12) Patent Application Publication
(10) Pub. No.: US 2022/0160810 A1
(43) Pub. Date: May 26, 2022

(54) METHODS FOR CONTINUOUS EXTRACTION AND PURIFICATION OF A UNIQUE FLAVAN-3-OL EXTRACT FROM IMMATURE WHOLE GRAPE CLUSTERS AND COMPOSITIONS THEREOF

(71) Applicant: E. & J. Gallo Winery, Modesto, CA (US)

(72) Inventor: James A. Kennedy, Modesto, CA (US)

(21) Appl. No.: 17/442,483

(22) PCT Filed: Mar. 16, 2020
(86) PCT No.: PCT/US2020/022958

§ 371 Date: Sep. 23, 2021

Related U.S. Application Data
(60) Provisional application No. 62/823,390, filed on Mar. 25, 2019.

(51) Int. Cl. A61K 36/87; A61K 31/353; C12P 17/06; B01D 15/42; A23L 33/105

(52) U.S. Cl. CPC A61K 36/87; A61K 31/353; C12P 17/06; B01D 15/424; A23L 33/105; A23V 2002/00; A61K 2236/331; A61K 2236/37; A61K 2236/55; A61K 2236/15; A61K 2236/53

(57) ABSTRACT

The present disclosure provides a novel process for the continuous extraction, purification and production of a flavan-3-ol extract from early harvested whole grape clusters. The final product from this process is particularly rich in monomeric and oligomeric flavan-3-ols and with an unusually high conversion of the final product into flavan-3-ol subunits under acid catalysis conditions. The oligomers and polymers in the final product are frequently referred to as proanthocyanidins in the field of polyphenol chemistry. The disclosure provides a novel process for the production of the final product via a continuous extraction process and with a final product having a unique composition.

### CROSS-REFERENCE TO RELATED APPLICATIONS

This application claims priority U.S. Provisional Application No. 62/823,390, filed Mar. 25, 2019, which is incorporated by reference herein in its entirety.

### TECHNICAL FIELD

The present disclosure relates to polyphenol chemistry products and processes.

### BACKGROUND

Flavonoids constitute an important group of dietary polyphenolic compounds that are widely distributed in plants. More than 4000 chemically unique flavonoids have been identified in plant sources, such as fruits, vegetables, legumes, nuts, seeds, herbs, spices, flowers, as well as in beverages such as tea, cocoa, beer, wine, and grape juice.

In the grape, the major flavonoid classes found include anthocyanins, flavonols and flavan-3-ols (including flavan-3-ol monomers as well as proanthocyanidins). Proanthocyanidins are oligomeric and polymeric compounds composed of flavan-3-ol subunits (FIGS. 1 and 2). These subunits include (+)-catechin, (−)-epicatechin, (−)-epigallocatechin and (−)-epicatechin 3-O-gallate. From a biological activity perspective, proanthocyanidin oligomers can be considered to have 2 to 7 subunits (dimers to heptamers); whereas polymers represent components with more than 7 subunits.

There is interest in utilizing flavan-3-ols from a commercial standpoint. In order to be used commercially as a grape extract, these compounds have to be produced in a more concentrated form. The general process in which the polyphenolic compounds are extracted, purified and concentrated from whole grapes, grape pomace and grape seeds is disclosed in commonly owned U.S. Pat. No. 6,544,581, entitled PROCESS FOR EXTRACTION, PURIFICATION AND ENRICHMENT OF POLYPHENOLIC SUBSTANCES FROM WHOLE GRAPES, GRAPE SEEDS AND GRAPE POMACE, which is incorporated herein by reference in its entirety. The processing methods reported in the literature for the production of grape extract from preveraison grapes do not exist.

### SUMMARY OF THE DISCLOSURE

The present disclosure provides a novel process for the continuous extraction, purification and production of a flavan-3-ol extract from early harvested whole grape clusters. The final product from this process is particularly rich in monomeric and oligomeric flavan-3-ols and with an unusually high conversion of the final product into flavan-3-ol subunits under acid catalysis conditions. The oligomers and polymers in the final product are frequently referred to as proanthocyanidins in the field of polyphenol chemistry. The disclosure provides a novel process for the production of the final product via a continuous extraction process and with a final product having a unique composition.

The disclosure generally encompasses methods for the extraction of flavan-3-ols from grape clusters and compositions thereof.

In one embodiment, the disclosure encompasses methods for the extraction of flavan-3-ols from grape clusters comprising:

i. adding the grape clusters to water to form a slurry the water is at a temperature above room temperature,

ii. adding the slurry to a decanter to separate the solids to form a crude extract;

iii. cooling the extract and treating the cooled extract with an enzyme;

iv. adding acid to form an acidified extract;

v. cooling the acidified extract for a time sufficient to allow macromolecules present in the extract to settle;

vi. filtering the extract to yield a clarified extract; and

vii. purifying the filtered extract.

In certain embodiments, the grape clusters are shredded.

In certain embodiments, the slurry is formed at a temperature above room temperature.

In certain embodiments, the enzyme is a pectolytic enzyme.

In certain embodiments, the purifying is done using adsorption-elution chromatography.

In certain embodiments, n the adsorption-elution chromatography is done on a XAD-7HP column.

Another embodiment encompasses methods for the continuous extraction of flavan-3-ols from grape clusters comprising:

i. shredding the grape clusters;

ii. adding the shredded grape clusters to water, wherein to form a slurry the water is at a temperature above room temperature,

iii. adding the slurry to a decanter separating the solids to form a crude extract;

iv. cooling the extract and treating the cooled extract with a pectolytic enzyme;

v. adding acid to form an acidified extract;

vi. cooling the acidified extract for a time sufficient to allow macromolecules present in the extract to settle;

vii. filtering the extract to yield a clarified extract; and

viii. purifying the filtered extract using adsorption-elution chromatography on a XAD-7HP column.

In another embodiment, the disclosure encompasses an polyphenolic composition comprising a total phenolic content (GAE, % w/w, dry basis) of greater that 90% comprising monomers/oligomers in an amount of about 70% and polymers in an amount of about 30%, wherein the monomers/oligomer content is 33.2% monomers, 6.9% dimers, 10.6% trimers, 9.0% tetramers, and 10.6% pentamers

In another embodiment, the composition is formulated into dietary supplements.

In another embodiment, the composition is a nutraceutical, food and/or beverage products.

In another embodiment, the pharmaceutical dosage forms, including capsules, tablets, powders, solutions, gels, suspensions, creams, pastes, gels, suppositories, or transdermal patches.

In another embodiment, the monomers are flavan-3-ols.

In another embodiment, the flavan-3-ols are epicatechin, catechin, epicatechin-3-O-gallate, and epigallocatechin.

In another embodiment, the flavan-3-ols is epicatechin.

In another embodiment, the flavan-3-ols is catechin.

In another embodiment, the flavan-3-ols is epicatechin-3-O-gallate.

In another embodiment, the flavan-3-ols is epigallocatechin.

In another embodiment, the composition is suitable for oral administration.

The present disclosure provides a novel process that allows for the continuous extraction of flavan-3-ols from whole grape clusters with subsequent purification and concentration into a spray dried powder. Aspects of several embodiments of the novel processes disclosed herein include continuous hot water extraction, enzyme treatment, pH treatment of the hot water extract, and the use of commercially available XAD-7HP adsorbent resin sold by The Dow Chemical Company, to maximize the concentration and purification of beneficial polyphenolic substances.

The processes of the present disclosure produce a highly concentrated flavan-3-ol product. Due to the timing of harvest (just prior to veraison) flavan-3-ol monomer, oligomer and polymer amounts in the grape have reached their maximum and are most easily extracted at this time which provides an opportunity to continuously extract the product. In addition, by extracting the material at this time, maximal preservation of flavan-3-ol structure is targeted.

The current disclosure does not require organic solvent extraction of the source materials, membrane filtration, or solvent-solvent partitioning. As such, the processes described in the present disclosure are safer, simpler and higher-yielding than those previously known. The present disclosure is thus better-suited for large scale commercial/industrial and winery production than previously known methods.

The product produced according to the present disclosure may be used in foods, beverages and nutraceuticals. From a biological activity perspective, the product in the present disclosure may help lower the incidence of cardiovascular diseases, may help prevent or treat prehypertension, metabolic syndrome, and may improve cognitive functions, slow progression of cognitive decline, and slow down damages to and instability within DNA with aging leading to cognitive decline. See for example: Spadafranca et al., “Effect of dark chocolate on plasma epicatechin levels, DNA resistance to oxidative stress and total antioxidant activity in healthy subjects”, British Journal of Nutrition, 103: 1008-14 (2010); Le et al., “Examining the impact of grape consumption on brain metabolism and cognitive function in patients with mild decline in cognition: A double-blinded placebo-controlled pilot study”, Experimental Gerontology, 87: 121-128 (2017).

In addition, due to the preservation of flavan-3-ol structure, it is expected that the product will be metabolized by the lower gut microflora to a greater extent. Seefor example: Appeldoorn et al., “Procyanidin dimers are metabolized by human microbiota with 2-(3,4-dihydroxyphenyl) acetic acid and 5-(3,4-dihydroxyphenyl-γ-valerolactone as the major metabolites”, Journal of Agricultural and Food Chemistry, 57: 1084-1092 (2009); Weise et al., “Comparative biokinetics and metabolism of pure monomeric, dimeric, and polymeric flavan-3-ols: A randomized cross-over study in humans”, Molecular Nutrition and Food Science, 59: 610-621 (2015); Alvarez-Cilleros et al., “Colonic metabolites from flavanols stimulate nitric oxide production in human endothelial cells and protect against oxidative stress-induced toxicity and endothelial dysfunction”, Food Chemistry and Toxicology, 115: 88-97 (2018); Ottaviani et al., “Evaluation at scale of microbiome-derived metabolites as biomarker of flavan-3-ol intake in epidemiological studies”, Scientific Reports, 8: 9859 (2018).

Antioxidant properties are beneficial across a wide range of applications. Thus, foods, beverages, dietary supplements, nutraceutical products and cosmetics containing the polyphenolic products according to the present disclosure may be produced. The products according to the present disclosures may be used in cosmetic preparations as an antioxidant for skin protection. Also, given the products unusually light color, it can be incorporated into beverages without imparting negative color.

### BRIEF DESCRIPTION OF THE DRAWINGS

FIG. 1 illustrates Flavan-3-ol subunits found in Vitis vinifera L.

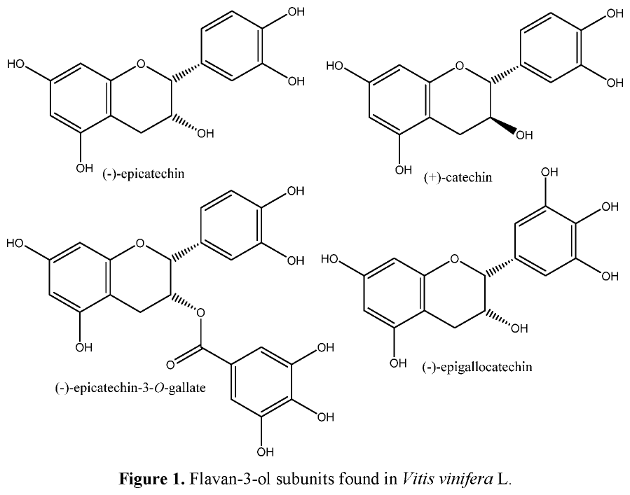

FIG. 2 illustrates general proanthocyanidin structure with predominant 4→8 interflavonoid linkage indicated.

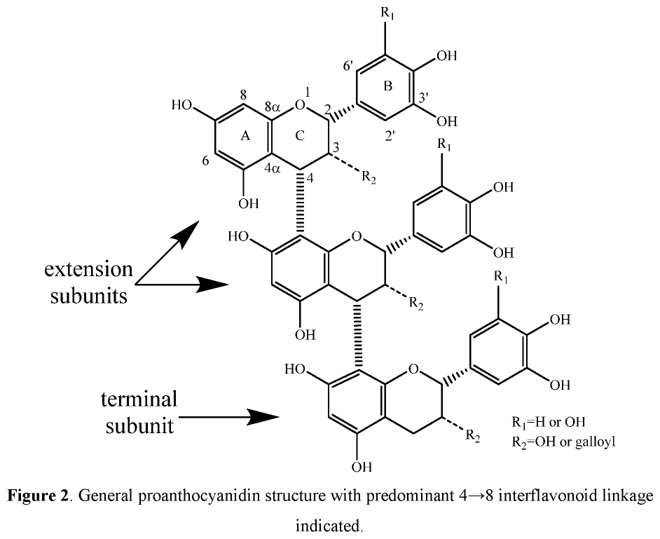

FIG. 3 illustrates a flow chart providing generalized extraction and purification process.

FIG. 4 illustrates a generalized schematic of berry development with flavan-3-ol production highlighted and harvest time indicated.

### DETAILED DESCRIPTION

As used throughout this specification, “flavan-3-ol monomer” or “monomer” refers to monomeric flavan-3-ol compounds such as (+)-catechin, (−)-epicatechin, (−)-epigallocatechin and (−)-epicatechin gallate. “Oligomeric proanthocyanidins” refers to compounds having a degree of polymerization (“DP”) of 2 to about 7; “polymeric proanthocyanidins” refers to procyanidins having a degree of polymerization of 8 or greater; “aqueous solvent” (e.g. “aqueous ethanol”) refers to a solution of water and solvent; “X % aqueous solvent” (e.g. “80% aqueous ethanol”) refers to a solution containing X % (v/v) of solvent. Thus, 80% aqueous ethanol contains 20% water and 80% ethanol (v/v).

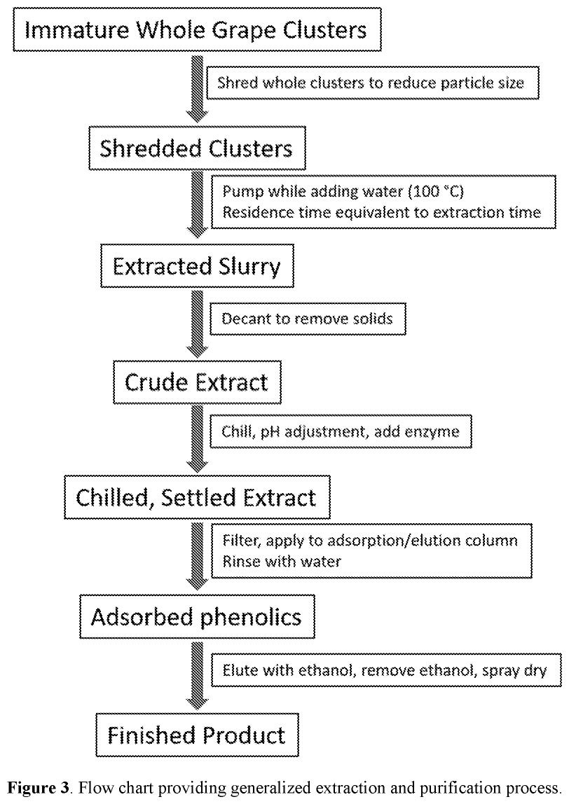

The instant disclosure provides a new process for the continuous extraction of whole grape clusters harvested prior to veraison and the production of a unique product containing a well preserved flavan-3-ol structure (FIG. 3).

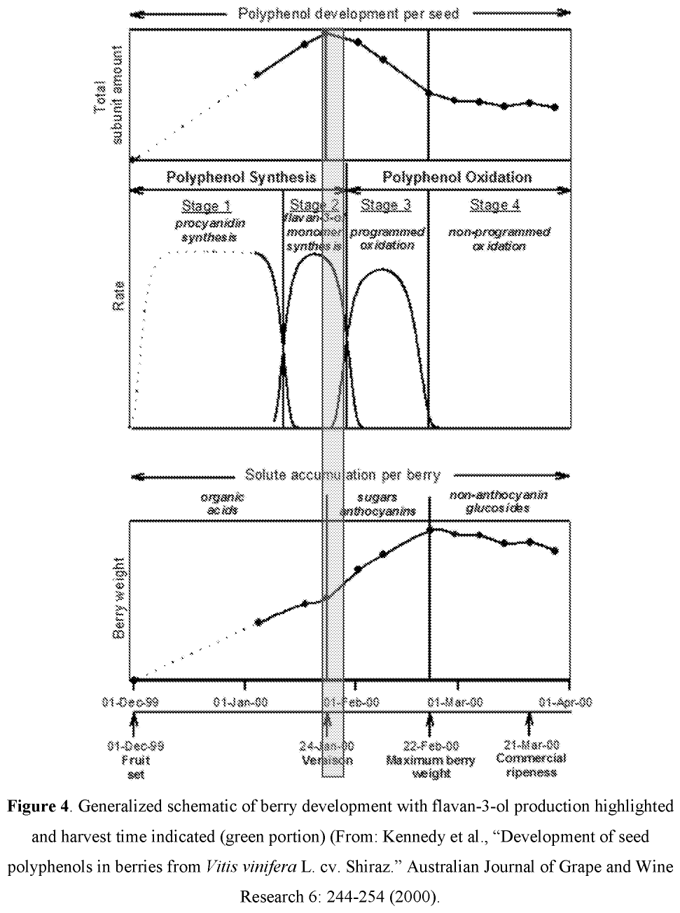

To extract whole grape clusters, the timing of harvest is critical so that maximal flavan-3-ol amount per berry is achieved. This time coincides with late lag phase/early veraison of berry maturity (FIG. 4). In certain embodiments, the whole grape clusters are shredded into approximate grape seed size and are then directed to a must pump where water (near 100° C.) is added. In certain embodiments, the combined grape/water slurry is pumped to a decanter, with a residence time equivalent to the extraction time (for example 5 minutes). In certain embodiments, a decanter is used to separate the gross solids from the crude extract.

In certain embodiments, the extract is cooled and treated with a suitable commercially available pectolytic enzyme, such as, for example, Pectinex® Ultra SP-L manufactured by Novo Nordisk, to break down cell wall constituents. In certain embodiments, the extract is enzyme-treated for a period of two hours at 80-120° F. In other embodiments, the extract may be enzyme-treated for 7-14 days or longer at about 40-50′ F.

In certain embodiments, the resulting extract is acidified with an acid, preferably a mineral acid, more preferably with sulfuric acid, to a pH of approximately 1.5-2.5 and allowed to react from 1 to 48 hours. In certain embodiments, the acidified extract is cooled for several weeks to allow for macromolecules, including proteins and polysaccharides, to settle. In certain embodiments, the cooled acidified extract is then filtered using diatomaceous earth to yield a clarified extract. Other filter aids such as perlite, may also be used.

In certain embodiments, the filtered extract is then purified by using adsorption-elution chromatography for example, on a XAD-7HP column including 1-100 bed volumes of the effluent is adsorbed onto the XAD-7HP column. In certain embodiments, the adsorption/elution process for the recovery of proanthocyanidins from the XAD-7HP column is similar to the process described in the U.S. Pat. No. 6,544,581 B1 for the concentration of grape seed proanthocyanidins.

In certain embodiments, the extract prepared in the final disclosure has unique properties when compared to a standard extract such as grape seed extract. In the example shown in Table 1, two grape seed extracts were analyzed and compared to the present disclosure. The extracts were all prepared using adsorption-elution chromatography and as such have high total phenols (>90% w/w, GAE). Among other places where the present disclosure is different is in the overall yield of proanthocyanidin-related subunits following acid-catalysis in the presence of excess nucleophile (72.2% w/w) relative to standard grape seed extracts (<55.0% w/w). In certain embodiments, the oligomeric composition is much higher than standard grape seed extracts. Taken together, the disclosure yields an extract that contains a higher proportion of low molecular weight flavan-3-ols of known composition. The composition of the disclosure can have a higher overall bioavailability due to its unique composition.

In certain embodiments, the dimers and trimers of the present disclosure had the activity of scavenging superoxide anion radicals. Superoxide anion radicals (O₂⁻) are a type of active oxygen formed within the living body, where the radicals not only exhibit sterilizing action but also induce an indiscriminate, strong oxidizing reaction. This effect is believed to cause conditions such as aging and tumor formation in the living body, typically through the peroxidation of unsaturated fatty acids in cell membranes (see, for example, NANZANDO'S MEDICAL DICTIONARY, 18th ed., p. 329, published Jan. 16, 1998). In addition, a peroxidation reaction of unsaturated fatty acids in food will lead to its deterioration and may even be involved in the emission of off-odor from the food. Therefore, the compounds of the present disclosure which can scavenge superoxide anion radicals have beneficial characteristics in that they can prevent a variety of conditions resulting from active oxygen including, for example, life-style related diseases such as hypertension, diabetes and hyperlipemia, cardiac diseases such as arteriosclerosis, and aging and cancer.

Therefore, the compounds of the present disclosure can be used in smaller amounts than the conventional superoxide anion radical scavengers of natural origin and can yet prevent a variety of conditions resulting from active oxygen including, for example, life-style related diseases such as hypertension, diabetes and hyperlipemia, cardiac diseases such as arteriosclerosis, and aging and cancer. In addition, since the compounds of the present disclosure are of natural origin, they feature high safety levels and can be ingested over a prolonged period to exhibit the intended efficacy.

The epigallocatechin dimers and/or trimers of the present disclosure may be incorporated in tea in order to potentiate the polyphenols in it, thereby producing foods and beverages that have not only the action of reducing neutral fat and preventing peroxidation of lipids, aging and obesity, but also the action of preventing a variety of conditions resulting from active oxygen including, for example, life-style related diseases such as hypertension, diabetes and hyperlipemia, cardiac diseases such as arteriosclerosis, and aging and cancer.

Examples of beverages in which the compounds of the present disclosure may be incorporated include soft drinks, tea beverages, liquid tonics, health drinks, nutrition supply drinks, sports drinks and carbonated drinks (including liquid concentrates and preparatory powders for these beverages), and exemplary foods in which the compounds may be incorporated include gums, candies, jellies, confectioneries in tablet form, health foods, nutrition supply foods, and dietary supplements.

In certain embodiments, the grape extracts of the present disclosure may be formulated into dietary supplements or pharmaceutical dosage forms, including capsules, tablets, powders, solutions, gels, suspensions, creams, pastes, gels, suppositories, transdermal patches, and the like. The dietary supplements in, for instance, powder or solution form, may be added to nutraceuticals, foods and/or beverages to form functional nutraceutical, food, and/or beverage products. In certain embodiments, the dietary supplements may be formulated as powders, for example, for mixing with consumable liquids such as milk, juice, water or consumable gels or syrups for mixing into other dietary liquids or foods. The dietary supplements of this disclosure may be formulated with other foods or liquids to provide pre-measured supplemental foods, such as single serving bars. Exemplary food products that may incorporate the grape extract of the present disclosure include dairy foods such as yogurt, cereals, breads, snack food products, fruit juices, soft drinks and other drinks. Flavorings, binders, protein, complex carbohydrates, vitamins, minerals and the like may be added as needed. Preferably, the grape extract is formulated for oral administration.

TABLE 1
Composition of the present disclosure versus traditional
grape seed extracts.

| | | Low | Mono- | High | Mono- |
|---|---|---|---|---|---|
| | Present | Grape | mer | Grape | mer |
| Extract Property | Disclosure | Extract | Seed | Extract | Seed |
| Conversion Yield | 72.2 | 35.0 | | 55.0 | |
| (% w/w)¹ | | | | | |
| Total Phenol | 91.9 | 90.0 | | 96.0 | |
| (GAE, % w/w, dry basis)² | | | | | |
| Size Distribution (HPLC)³ | | | | | |
| Total HPLC Peak Area | 70954 | 56595 | | 92639 | |
| Polymers | 29.6 | 50.0 | | 55.5 | |
| (% Area, >5mers) | | | | | |
| Oligomers | 70.3 | 50.0 | | 44.5 | |
| (% Area, 1-5mers) | | | | | |
| Monomers | 33.2 | 15.3 | | 13.9 | |
| Dimers | 6.9 | 8.0 | | 6.0 | |
| Trimers | 10.6 | 10.1 | | 9.9 | |
| Tetramers | 9.0 | 8.9 | | 8.2 | |
| Pentamers | 10.6 | 7.6 | | 6.5 | |

Notes:
¹Conversion of extract into known flavan-3-ol subunits (w/w), as described in Kennedy and Jones, “Analysis of proanthocyanidin cleavage products following acid-catalysis in the presence of excess phloroglucinol”, Journal of Agricultural and Food Chemistry 49: 1740-1746 (2001).
²Total phenolic content (GAE, w/w) based upon response to Folin-Ciocalteu reagent, as described in Singleton et al., “Analysis of total phenols and other oxidation substrates and antioxidants by means of Folin-Ciocalteu reagent”, Oxidants and Antioxidants, Pt. A, 299:152-178 (1999).
³Composition of extracts based upon peak area size, as described in Kelm et al., “High-performance liquid chromatography separation and purification of cacao (Theobroma cacao L.) procyanidins according to degree of polymerization using a diol stationary phase” 54: 1571-1576 (2006).

The present disclosure also provides polyphenolic products such as foods, beverages, dietary supplements, nutraceutical products, cosmetics and pharmaceutical dosage forms containing the polyphenolic products or grape extracts according to the present disclosure. In certain embodiments, the products produced according to the present disclosure may be used in foods, beverages and nutraceuticals as an antioxidant. In certain embodiments, the products produced according to the present disclosure may be administered to a subject, such as an animal or a human. In certain embodiments, the products according to the present disclosure may help lower the incidence of cardiovascular diseases, may help prevent or treat prehypertension, metabolic syndrome, and may improve cognitive functions, slow progression of cognitive decline, and slow down damages to and instability within DNA with aging leading to cognitive decline.

Recent studies involving the preservation of telomeres and DNA integrity showed that cocoa extract (proanthocyanidins) slowed the shortening of telomeres. Damages to and instability within DNA as people age is likely a factor in many diseases or conditions of aging, including cognitive decline. See for example: Spadafranca et al., “Effect of dark chocolate on plasma epicatechin levels, DNA resistance to oxidative stress and total antioxidant activity in healthy subjects”, British Journal of Nutrition, 103: 1008-14 (2010). Therapeutic benefits of grape consumption were shown in preserving brain function in individuals experiencing mild cognitive changes, which is especially important given the increasing percentage of adults who will develop dementia due to rising elderly population. Polyphenols may also modulate brain function differently depending on their subtype limiting discernment of which class contributed most significantly to the neuroprotective effects. Significant protection from longitudinal changes in cerebral metabolism, which in turn were correlated with improvement in attention/working memory performances, is consistent with a beneficial effect of daily intake of grapes with respect to preservation of metabolic activity in individuals experiencing mild cognitive decline. See for example: Lee et al., “Examining the impact of grape consumption on brain metabolism and cognitive function in patients with mild decline in cognition: A double-blinded placebo controlled pilot study”, Experimental Gerontology, 87: 121-128 (2017); Nofar, WO2014141265 A1—“Inhibition ofneurodegenerative disease by grape seed extract, green tea, and probiotic bacteria”; Lamport et al., “The effect of flavanol-rich cocoa on cerebral perfusion in healthy older adults during conscious resting state: a placebo controlled, crossover, acute trial”, Psychopharmacology, 232: 3227-3234 (2015); Rendeiro et al., “The mechanisms of action of flavonoids in the brain: Direct versus indirect effects”, Neurochemistry International, 89: 126-139 (2015).

In certain embodiments, the products according to the present disclosure can be metabolized by the gut microflora to produce metabolites that are absorbed and have the same activities as described above. Due to the preservation of flavan-3-ol structure, it is expected that the products according to the present disclosure may be metabolized by the lower gut microflora to a greater extent than a standard grape seed extract. See for example: Appeldoorn et al., “Procyanidin dimers are metabolized by human microbiota with 2-(3,4-dihydroxyphenyl) acetic acid and 5-(3,4-dihydroxyphenyl-γ-valerolactone as the major metabolites” Journal of Agricultural and Food Chemistry, 57: 1084-1092 (2009); Weise et al., “Comparative biokinetics and metabolism of pure monomeric, dimeric, and polymeric flavan-3-ols: A randomized cross-over study in humans”, Molecular Nutrition and Food Science, 59: 610-621 (2015); Alvarez-Cilleros et al., “Colonic metabolites from flavanols stimulate nitric oxide production in human endothelial cells and protect against oxidative stress-induced toxicity and endothelial dysfunction.” Food Chemistry and Toxicology, 115: 88-97 (2018); Ottaviani et al., “Evaluation at scale of microbiome-derived metabolites as biomarker of flavan-3-ol intake in epidemiological studies”, Scientific Reports, 8: 9859 (2018).

In certain embodiments, the products according to the present disclosures may be used to improve cognitive functions in a healthy subject. In certain embodiments, the products according to the present disclosures may be used in cosmetic preparations as an antioxidant for skin protection.

In certain embodiments, the utility of the grape extract may be tested using the methods known to a skilled artisan. See for example: Wang et al., “Brain-targeted proanthocyanidin metabolites for Alzheimer's Disease treatment”, Journal of Neuroscience, 32: 5144-5150 (2012); Hayden et al., “Inhibiting amyloid β-protein assembly: Size-activity relationships among grape seed-derived polyphenols”, Journal of Neurochemistry, 135: 416-430 (2015); Lamport et al., “The effect of flavanol-rich cocoa on cerebral perfusion in healthy older adults during conscious resting state: A placebo controlled, crossover, acute trial”, Psychopharmacology, 232: 3227-3234 (2015); Brickman et al., “Enhancing dentate gyrus function with dietary flavanols improves cognition in older adults” Nature Neuroscience, 17: 1798-1803 (2014); Appeldoorn et al., “Procyanidin dimers are metabolized by human microbiota with 2-(3,4-dihydroxyphenyl) acetic acid and 5-(3,4-dihydroxyphenyl-γ-valerolactone as the major metabolites.” Journal of Agricultural and Food Chemistry, 57: 1084-1092 (2009); Weise et al., “Comparative biokinetics and metabolism of pure monomeric, dimeric, and polymeric flavan-3-ols: A randomized cross-over study in humans”, Molecular Nutrition and Food Science, 59: 610-621 (2015); Alvarez-Cilleros et al., “Colonic metabolites from flavanols stimulate nitric oxide production in human endothelial cells and protect against oxidative stress-induced toxicity and endothelial dysfunction”, Food Chemistry and Toxicology, 115: 88-97 (2018); Ottaviani et al., “Evaluation at scale of microbiome-derived metabolites as biomarker of flavan-3-ol intake in epidemiological studies”, Scientific Reports 8: 9859 (2018).

In certain embodiments, the polyphenolic products or grape extracts according to the present disclosure may be administered to a subject daily or as needed, in an amount effective to achieve a therapeutic or beneficial effect on the subject. The dose and/or dose frequency may vary depending on factors such as the age of the subject, the body weight of the subject, the severity of the disease or condition that the polyphenolic products is used to treat or prevent, and the route of administration. In general, the total daily dose range may be from about 100 mg to about 1000 mg grape extract, administered in single or divided doses. An oral daily dose range is preferably from about 100 mg to about 1000 mg of the grape extract (i.e., excluding excipients and carriers). For example, capsules or tablets may be formulated in either 100 mg or 1000 mg doses, whereas beverages may be formulated with the targeted dose of grape extract of the present disclosure.

In certain embodiments, the polyphenolic products of the present disclosure may be formulated in a conventional manner (i.e. by dry mixing, dry or wet granulation, direct compression), in admixture with pharmaceutically acceptable carriers, excipients, vitamins, minerals and/or other nutrients. Representative carriers and excipients include, but are not limited to, starches, sugars, microcrystalline cellulose, diluents, granulating agents, lubricants, binders, disintegrating agents, and the like, in the case of oral solid preparations (such as powders, capsules, and tablets).

In certain embodiments, any suitable route of administration may be employed to administer the dietary supplements of the disclosure to an individual. Suitable routes include, for example, oral, rectal, parenteral, intravenous, topical, transdermal, subcutaneous, nasal, and intramuscular. Although any suitable route of administration may be employed for providing the patient with an effective amount of the grape extract according to the methods of the present disclosure, oral administration is preferred, including solid dosage forms such as tablets, capsules, or powders. It is also preferred that the grape extract is formulated for use in functional nutraceutical, food, or beverage products.

In certain embodiments, the grape extract according to the present disclosure can also be combined with other active agents, including but not limited to Curcumin (e.g., as absorption-enhanced BMC95®; e.g., 400-800 mg daily), R-Lipoic acid (e.g., 240-480 mg daily), Acetyl-L-Carnitine (e.g., 1,000-3,000 mg daily), Fish oil (e.g., providing 1,400 mg EPA and 1,000 mg DHA daily), Vinpocetine (e.g., 10-30 mg daily), Pyrroloquinoline quinone (PQQ) (e.g., 10-20 mg daily), Phosphatidylserine (e.g., 100 mg daily), Coffee (caffeinated; e.g., 3-5 cups daily, ideally standardized to provide highest concentration of polyphenols), Blueberry extract (e.g., 150-750 mg daily), Green tea extract (e.g., standardized to 98% polyphenols; e.g., 725-1,450 mg daily), Resveratrol (e.g., 250 mg daily), Whole grape extract (e.g., 150 mg daily), Magnesium (e.g., 140 mg daily as magnesium-L-threonate and at least 100 mg daily as magnesium citrate), Vitamin B12 (e.g., 1,000-5,000 mcg daily), Vitamin B6 (e.g., 250 mg daily), Folate (preferably as L-methylfolate; e.g., 400-1,000 mcg daily), Vitamin D (e.g., 5,000-8,000 IU daily; optimal blood levels of 25-OH-vitamin D are between 50-80 ng/mL), Coenzyme Q10 (preferably ubiquinol; e.g., 100-300 mg daily), N-acetylcysteine (NAC; e.g., 600-1,800 mg daily), Ashwagandha extract (e.g., 250 mg daily), Alpha glyceryl phosphoryl choline (e.g., 600 mg daily), Huperzine A (e.g., 200-800 mcg daily), Panax ginseng (e.g., 400-1,000 mg daily), Vitamin E (e.g., 400 IU daily with at least 200 mg gamma tocopherol), and Ginkgo biloba (standardized extract; e.g., 120-240 mg daily).

While the claimed disclosure has been described in detail and with reference to specific embodiments thereof, it will be apparent to one of ordinary skill in the art that various changes and modifications can be made to the claimed disclosure without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this disclosure, and are covered by the following claims.

What is claimed is:

1. A method for the extraction of flavan-3-ols from grape clusters comprising:
i. adding the grape clusters to water to form a slurry the water is at a temperature above room temperature,
ii. adding the slurry to a decanter to separate the solids to form a crude extract;
iii. cooling the extract and treating the cooled extract with an enzyme;
iv. adding acid to form an acidified extract;
v. cooling the acidified extract for a time sufficient to allow macromolecules present in the extract to settle;
vi. filtering the extract to yield a clarified extract; and
vii. purifying the filtered extract.

2. The method of claim 1, wherein the grape clusters are shredded.

3. The method of claim 1, wherein the slurry is formed at a temperature above room temperature.

4. The method of claim 1, wherein the enzyme is a pectolytic enzyme.

5. The method of claim 1, wherein the purifying is done using adsorption-elution chromatography.

6. The method of claim 5, wherein the adsorption-elution chromatography is done on a XAD-7HP column.

7. A method for the continuous extraction of flavan-3-ols from grape clusters comprising:
i. shredding the grape clusters;
ii. adding the shredded grape clusters to water, wherein to form a slurry the water is at a temperature above room temperature,
iii. adding the slurry to a decanter separating the solids to form a crude extract;
iv. cooling the extract and treating the cooled extract with a pectolytic enzyme;
v. adding acid to form an acidified extract;
vi. cooling the acidified extract for a time sufficient to allow macromolecules present in the extract to settle;
vii. filtering the extract to yield a clarified extract; and
viii. purifying the filtered extract using adsorption-elution chromatography on a XAD-7HP column.

8. An polyphenolic composition comprising a total phenolic content (GAE, % w/w, dry basis) of greater that 90% comprising monomers/oligomers in an amount of about 70% and polymers in an amount of about 30%, wherein the monomers/oligomer content is 33.2% monomers, 6.9% dimers, 10.6% trimers, 9.0% tetramers, and 10.6% pentamers

9. The composition of claim 8, wherein the composition is formulated into dietary supplements.

10. The composition of claim 8, wherein the composition is a nutraceutical, food and/or beverage products.

11. The composition of claim 8, wherein pharmaceutical dosage forms, including capsules, tablets, powders, solutions, gels, suspensions, creams, pastes, gels, suppositories, or transdermal patches.

12. The composition of claim 8, wherein the monomers are flavan-3-ols.

13. The composition of claim 12, wherein the flavan-3-ols are epicatechin, catechin, epicatechin-3-O-gallate, and epigallocatechin.

14. The composition of claim 12, wherein the flavan-3-ols is epicatechin.

15. The composition of claim 12, wherein the flavan-3-ols is catechin.

16. The composition of claim 12, wherein the flavan-3-ols is epicatechin-3-O-gallate.

17. The composition of claim 12, wherein the flavan-3-ols is epigallocatechin.

18. The composition of claim 8 suitable for oral administration.
